(19) United States
(12) Patent Application Publication
(10) Pub. No.: US 2016/0120471 A1
(43) Pub. Date: May 5, 2016

(54) PIECE OF CLOTHING FOR A HUMAN BEING

(71) Applicant: Gerd Hexels, Nettetal (DE)

(72) Inventor: Gerd Hexels, Nettetal (DE)

(73) Assignee: HEXONIA GMBH, NETTETAL (DE)

(21) Appl. No.: 14/956,705

(22) Filed: Dec. 2, 2015

Related U.S. Application Data
(63) Continuation of application No. 13/509,858, filed on Jun. 8, 2012, now abandoned.

(51) Int. Cl. A61B 5/00; A61B 5/103; A61B 5/0205; F41H 1/02; A41D 1/00

(52) U.S. Cl. CPC A61B 5/6804; F41H 1/02; A41D 1/002; A61B 5/02055; A61B 5/103; A61B 5/021

(57) ABSTRACT

A piece of clothing is described and which detects external effects, and which further generates a signal which can be relayed to an analysis unit, and a multiplicity of sensors are incorporated into the piece of clothing, and which are arranged in zones of the piece of clothing, and which further adjoins a vital organ of a human body wearing the piece of clothing.

The present patent application claims priority from German Patent application. Serial No. 10 2009 046 861.7 and which was filed on Nov. 19, 2009, and PCT/EP2010/065441, and which was filed on Oct. 14, 2010.

The present invention relates to a piece of clothing for a human being, and having sensors which detect external effects and generate a signal which can be relayed to an analysis unit.

A piece of clothing according to the species as mentioned above is known from U.S. Pat. No. 6,315,009 B1.

In particular in the case of military and police actions, humans are subjected to the danger of being injured, possibly in a life-threatening or even deadly manner. It can be particularly advantageous if a central headquarters or the like, is informed early that a person involved in such an action has been injured. Furthermore, it can be significant for rescuing or aiding the person if rescue workers, for example, paramedics, are already informed about the type of injury and possibly how severe the injury is before they arrive at the injured person.

The solution known from the teachings of U.S. Pat. No. 6,315,009 B1 provides that a piece of clothing, for example, in the form of a shirt, is provided with optical sensors. These can be constructed as optical fibers, or optical waveguides, and which are fabricated from quartz plastic or silicone. In this arrangement, a signal is generated if a projectile breaks through the optical fiber. An analysis unit is informed by the signal that the optical waveguide is interrupted.

Furthermore, reference is made to U.S. Pat. No. 6,687,523 B1 regarding additional prior art.

The known prior art, in particular in U.S. Pat. No. 6,315,009 B1 and U.S. Pat. No. 6,687,523 B1 has a known disadvantage in that a signal can only be generated which informs about the fact that the optical waveguide has been broken through. The signal, however, contains no information about where a projectile or a stabbing weapon, for example, has penetrated into the human body who is wearing the clothing. The rescue workers therefore accordingly arrive unprepared at the injured person. Furthermore, it is quite disadvantageous that an injury by an impact, which can possibly also result in severe injuries (trauma) up to and leading to the death of the affected person, is not readily detected.

The present invention therefore finds novelty in providing a piece of clothing for a human being, and which allows injuries on the human body, caused by stabbing weapons or firearms, to be recognized reliably, and which further allows the injury to be assigned or identified, as precisely as possible, to an affected vital organ.

This object is achieved according to the invention as disclosed in the pending claims.

It is presumed in the following description that the piece of clothing as described, is worn correctly, and in the intended manner.

Because the sensors with which the piece of clothing is provided are implemented as electrical circuits, and/or as elements whose sensor property is based, at least in whole, or in part, on the piezo effect, and further, the sensors are arranged in at least one zone of the piece of clothing, and which adjoins vital organs, it may be established, comparatively precisely, and in relative comparison with the known prior art, at which point of the body the person wearing the piece of clothing was affected. According to the earlier prior art teachings, it could only be established that an optical waveguide had been interrupted. A more precise assignment of the location of the interruption was not possible.

It is additionally advantageous in the solution as provided according to the teachings of the present invention that multiple gunshot wounds be recognized or recorded.

In the case of an embodiment of the sensors as employed in the invention and which utilize electrical circuits or electrical printed conductors, the position at which the circuit or the printed conductor is interrupted may be established using known technical means. Through the knowledge of the interruption location on the piece of clothing, conclusions can then be drawn about the region of the human body which was affected by a projectile or a stabbing weapon. Alternatively, or additionally, a plurality of small electrical circuits or networks can also be used, so that in the case of a failure of a circuit, conclusions about the injury location are possible on the basis of its position on the piece of clothing.

It is advantageous if the electrical circuits have printed conductors extending in the form of waves or curves. The inventor has recognized that it is advantageous to arrange the printed conductors elastically so that they are not torn off by a pulling movement of the piece of clothing. Such an arrangement may be achieved particularly advantageously if the printed conductors extend in the form of waves or curves, so that a pulling or stretching movement of the printed conductors is made possible.

It has also proven to be advantageous if the printed conductors extend in the form of waves or curves in the case of an implementation of the printed conductors by utilizing an electrically conductive polymer which is described in greater detail hereinafter. In the present invention, an electrically conductive polymer is utilized in a specific design of an electrical circuit, so that the features described with respect to the electrical circuit always also apply for the electrically conductive polymer.

It is particularly advantageous if the electrical circuits or the printed conductors are imprinted, or printed directly onto, the piece of clothing. It can also be provided that the electrical circuits or the printed conductors are printed onto a textile, which is subsequently connected to, or made integral with, the piece of clothing in a known manner.

The sensors which are implemented in the invention as elements, whose sensor property is based on the piezo effect, are referred to, hereafter, as piezo elements for short.

A piezo element is a component which utilizes the so-called piezo effect, in order, for example, to generate an electrical voltage upon the application or action of a mechanical force (e.g., pressure). Piezo elements can be specific crystals (piezo crystals) or piezoelectric ceramics, i.e., polycrystalline materials. A particularly suitable material can be lead-zirconate-titanate.

The implementation of the sensors, as discussed above, as piezo elements has the advantage that they respond to pressure, and it is therefore possible to detect an impact which is executed against the person who wears the piece of clothing. It is therefore not only possible to establish that a gunshot or stabbing injury has occurred, but also hazardous impacts. An implementation of piezo elements, for example, in head covers, to detect impacts which are particularly hazardous, or in the heart region, can be particularly advantageous. In addition, it can be advantageous if the piezo elements are used jointly with sensors based on electrical circuits. Therefore, both a stabbing or gunshot injury, and also an injury by an impact are thus recognized in a particularly advantageous fashion.

Additionally, not only impacts, but also pressure waves, occasioned by explosive detonations, and the like, can be recognized by the piezoelectric embodiment of the sensors as described hereinafter.

Instead of piezo elements, other known pressure sensors can also be used. In the present case, the term “piezo element”, in its most general meaning, is therefore also intended to comprise pressure sensors of other designs.

It is advantageous if the sensors, i.e., the electrical circuits or the piezo elements, be utilized in, or with multiple zones, which are assigned to various vital organs.

Through the implementation of the sensors in multiple zones, an undesired effect on the human body can be readily recognized particularly advantageously. For example, the vital organs, to which corresponding zones are assigned, can preferably be the heart, the lungs, the stomach, the intestines, the liver, or the kidneys. These zones are preferably implemented both on the front side, and also on the rear side of the piece of clothing. These zones can mutually overlap or can be arranged in an interleaved pattern. Furthermore, it can be advantageous if the aforementioned zones, as a whole, at least approximately, and preferably completely, encompass the piece of clothing, so that the entire piece of clothing does not have any region which is not monitored.

Through the front and rear application of the system or the zones, in contrast to the teachings of the optical waveguide technology as known from the prior art, gunshot wounds which completely pass through a user's body can also be recognized.

The aforementioned zones can be adapted to the anatomical and organ-specific arrangements in the human body. For example, in the case of an implementation of the zone by an electrical circuit, if the zone is destroyed by a gunshot, for example, the circuit is interrupted. A message can thus be generated via a control circuit, to an analysis unit. Information about the status of the wearer of the piece of clothing can then, optionally, be transmitted continuously via radio to a monitoring station.

To transmit the signal, a radio unit can be integrated into the piece of clothing or a connection to an external radio unit can be provided. Furthermore, an interface, preferably a USB interface, can be provided so as to read-out the signals provided by sensors and/or the analysis unit.

The signal can be transmitted by the radio unit continuously, at intervals, or when the sensors have detected an external action.

It is also advantageous, and a feature of the present invention if vital sensors are provided, which preferably detect various vital body signs such as the heartbeat; pulse; respiratory frequency; oxygen saturation in the blood; body temperature and/or the blood pressure. This vital body information can also be relayed to an analysis unit or to a monitoring unit. In the case of a detection of an impact or a stabbing or gunshot injury, it can thus be recognized rapidly whether the external effect on the human body is life-threatening. The required rescue or first aid measures can then be coordinated accordingly. In addition, by detecting the vital functions, incorrect messages can be reduced. It can optionally be provided that the ascertainment of the vital body functions does not occur continuously, but rather is triggered by a radio command. For example, a blood pressure measurement could be triggered by a radio command.

A temperature measurement is particularly advantageous, because the temperature of a human body typically rises when it is injured by an impact or serious wound or injury. The detection of an impact, in combination with an increase of the body temperature, can therefore be helpful for indicating a possibly life-threatening situation.

To ascertain the heartbeat, for example, it can be provided that a corresponding first vital organ sensor is integrated in the piece of clothing in the region which adjoins the heart. Furthermore, for example, to ascertain the respiratory frequency, a corresponding second vital organ sensor can be integrated in the piece of clothing in the region of the lungs.

The above-mentioned sensors, based on the piezo elements, can be connected in an arbitrary manner to the piece of clothing. An implementation of the piezo elements as a piezo film is particularly preferable. This can be connected to the piece of clothing in a simple manner.

It can be advantageous if the sensors based on the electrical circuits are implemented as printed circuits. The circuits can be printed on films or on other flexible materials.

The sensors, in particular the electrical circuits, can also be stitched or woven into the piece of clothing.

If it is provided that the piezo elements are used jointly with electrical circuits, it can be provided that the sensor types which are used are applied or integrated in two layers, preferably one over another, in the piece of clothing. It is also suitable for this purpose to integrate the respective sensor types in films. It can optionally be provided that the piece of clothing is only provided with two sensor types at the points at which it adjoins impact-sensitive regions of the body.

The piece of clothing according to the present invention can be implemented in an arbitrary manner, that is, preferably for a human upper body for example. The piece of clothing can also be implemented as underclothing, as a shirt or T-shirt, as an overall, as a typical upper piece of clothing, as a jacket, as a head cover, or the like. An implementation of the piece of clothing as trousers is also fundamentally possible. It is advantageous if the piece of clothing is breathable and/or windproof and/or watertight and/or water-vapor permeable. It is particularly preferable if the piece of clothing has all of the above-mentioned properties.

It is advantageous if the sensors, in their implementation as elements whose sensor property is based on the piezo effect, are implemented as a polymer. It is particularly preferable if the polymer is a polyvinylidene fluoride (PVDF). The polyvinylidene fluoride can preferably have piezoelectric properties through polarization. The polyvinylidene fluoride is preferably implemented as a film or as a layer.

It is advantageous if the aforementioned polyvinylidene fluoride is applied in a honeycomb structure to the piece of clothing or has a honeycomb structure. The positive clothing-physiological properties of the piece of clothing are thus retained. A pressure induced by an impact, or a ballistic impact on the piece of clothing always affects a larger area than a point of the honeycomb structure has. An impact or a ballistic impact will therefore not pass between a honeycomb structure, and can therefore be recognized by the piezoelectric properties of the polyvinylidene fluoride.

In one embodiment of the invention, it can be provided that the polyvinylidene fluoride is arranged in at least one zone of the piece of clothing. The polyvinylidene fluoride is preferably implemented on the front side, and/or the rear side of the piece of clothing. The piece of clothing is preferably completely, or at least approximately completely provided with a layer of the polyvinylidene fluoride, preferably with a layer which is formed by a honeycomb structure.

A conductor structure or printed conductors can be applied to the layer made of the polyvinylidene fluoride (PVDF). The conductor structure or the printed conductors can therefore relay or absorb the pulses of the polyvinylidene fluoride. Various possibilities are known from the prior art for implementing the conductor structure or printed conductors. For example, the conductor structure, or the printed conductors can be applied by an exposure and etching technology employing copper.

The conductor structure or the printed conductors can also be coated with a breathable coating and/or a corresponding film, in particular to prevent oxidation. This is advantageous if the conductor structure or the printed conductors are copper conductors.

The implementation of the printed conductors and conductor structures can be performed on arbitrary substrates, preferably in a meandering technique.

Furthermore, it is advantageous if the printed conductors or the conductor structure have connection points for attaching further sensors, and in particular vital body sensors, as were already described above. Measurements, for example, of the pulse or other vital body functions, can thus be performed at arbitrary intervals, and locations.

It is advantageous if the power supply of the system is performed by film batteries, which are preferably printed on the garment.

A particularly advantageous implementation of the printed conductors and the conductor structure or an advantageous possibility in general for transmitting pulses or other signals is to apply a further electrically conductive polymer, for example, based on polyethylene dioxythiopene (PEDOT), to the polymer having the piezoelectric properties. The polyethylene dioxythiopene is preferably applied to a layer or an area or a film which is formed by a polyvinylidene fluoride. A polyethylene dioxythiopene is an electrically conductive polymer which is used in the prior art for flexible displays, flexible solar cells, and so-called organic light emitting diodes (LEDs).

The electrically conductive polymer which is applied to the polymer having the piezoelectric properties can register the destruction or damage of the system, which is occasioned by a gunshot, for example. The electrically conductive polymer can preferably take over the function of the ballistic conductor, i.e., the function which was already described, above, with respect to the electrical circuit. The electrically conductive polymer can therefore preferably fulfill the functions which the electrical circuit fulfills. The above-described features, with respect to the electrical circuit therefore also applies to the electrically conductive polymer, and in particular to the polyethylene dioxythiopene, especially if it is provided in the form of printed conductors or a conductor structure.

A circuit can be provided according to the teachings of present invention, and which, on the one hand, recognizes pulses of the polyvinylidene fluoride via the electrically conductive polymers and, on the other hand, recognizes a destruction of the printed conductors formed by the electrically conductive polymer, and preferably the polyethylene dioxythiopene. This can preferably be performed in that, on the one hand, a voltage is built up to record the pulses of the piezoelectric polymer and, on the other hand, a voltage is applied to recognize whether the printed conductors are interrupted or destroyed.

According to the teachings of the present invention, polyester knitwear, or the like, can be coated to form a network with the polyvinylidene fluoride as a piezo sensor. Printed conductors, preferably made of a polyethylene dioxythiopene, and preferably disposed, in a meandering form, can then be printed on the garment, and above, or below, the coating.

A particularly advantageous embodiment of a piece of clothing comprises implementing it as a soft-ballistic or hard-ballistic protective vest. Such soft-ballistic or hard-ballistic protective vests are well known from the general prior art. Such protective vests are typically worn by endangered persons, for example, by soldiers or by police officials. It is particularly advantageous especially in this case if external effects on the human body are recognized. The protective vests, which are also referred to as bulletproof vests, can have soft-ballistic and/or hard-ballistic inserts on their front and/or rear sides. It can be advantageous if only the ballistic packets (soft-ballistic or hard-ballistic) are coated with sensors, for example, a film circuit membrane. In the event of a destruction of the ballistic protective packets, a signal effect is therefore generated simultaneously. Vital functions can also be scanned and biosensors can be integrated within the system in this case.

In the case of an embodiment of the piece of clothing as a protective vest, it is advantageous if the sensors are arranged on the side of the protective vest facing toward the body. The arrangement can occur on the side of the ballistic protective packets of the protective vest facing toward the body. It is advantageous if the sensors are applied to, or integrated in, the external sheath of the soft-ballistic or hard-ballistic protective packets. It can additionally be advantageous if the sensors, in particular in an embodiment as electrical circuits, are arranged on a protective film, such as a UV protective film of the ballistic protective packets. In particular the soft-ballistic protective packets typically have a protective film in any case, which is to protect it against undesired impairments, for example, due to UV radiation, or the penetration of water. The sensors can then be applied to these same protective films.

It is advantageous if the protective vest is provided with a computer unit for analyzing the signals, or alternatively a connection to a computer unit exists.

Well-equipped military personnel currently carry protective vests, in which corresponding computer units are already integrated.

Advantageous embodiments and refinements of the invention result from the further dependent claims. An exemplary embodiment is schematically illustrated hereafter.

In the figures:

FIG. 1 shows a top view of a piece of clothing according to the teachings of the present invention and having multiple zones shown as examples, which are monitored by schematically illustrated sensors;

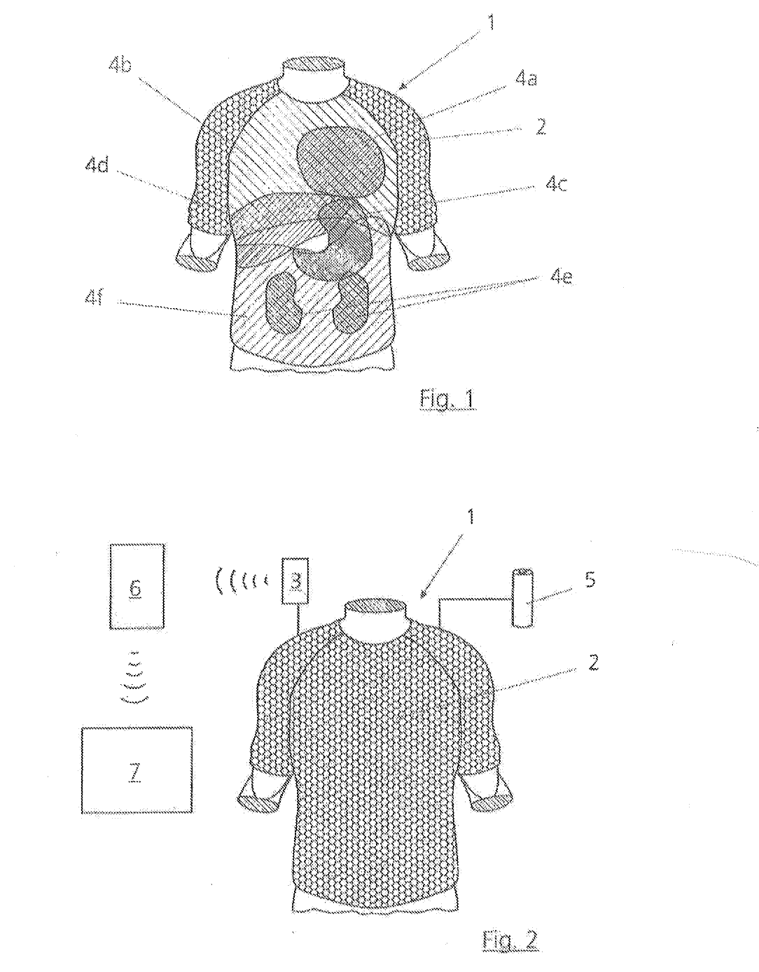

FIG. 2 shows a piece of clothing according to the teachings of the present invention, and which is completely provided with schematically illustrated sensors;

FIG. 3 shows a piece of clothing according to the present invention, and having a schematic illustration of the attachment of the monitored zones to a radio unit and a further interface;

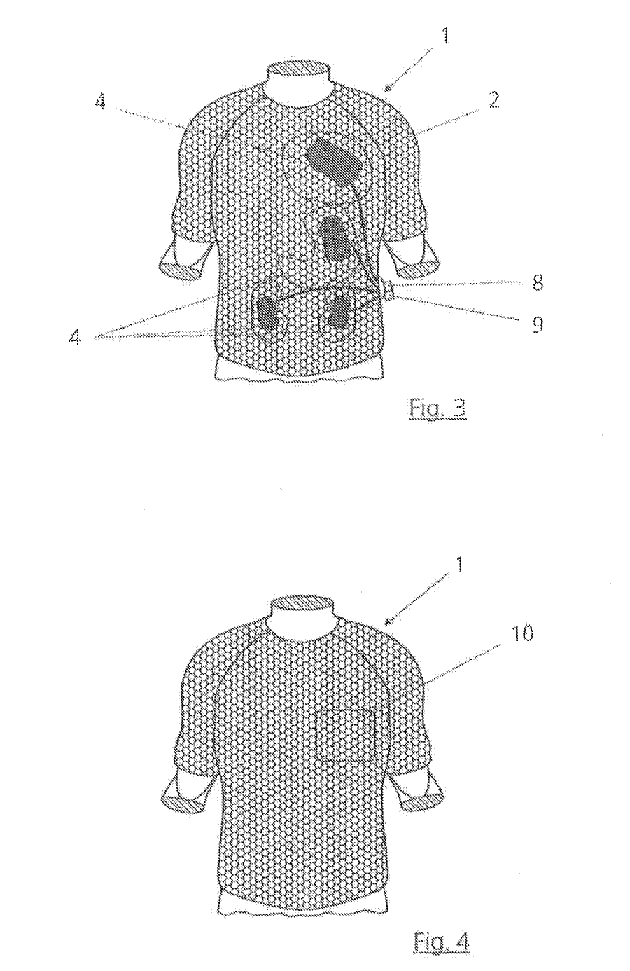

FIG. 4 shows a piece of clothing according to the present invention, and having a schematic illustration of a vital sensor in the region of a heart of a wearer of the piece of clothing;

FIG. 5 shows a schematic illustration of a protective vest which may incorporate features of the present invention;

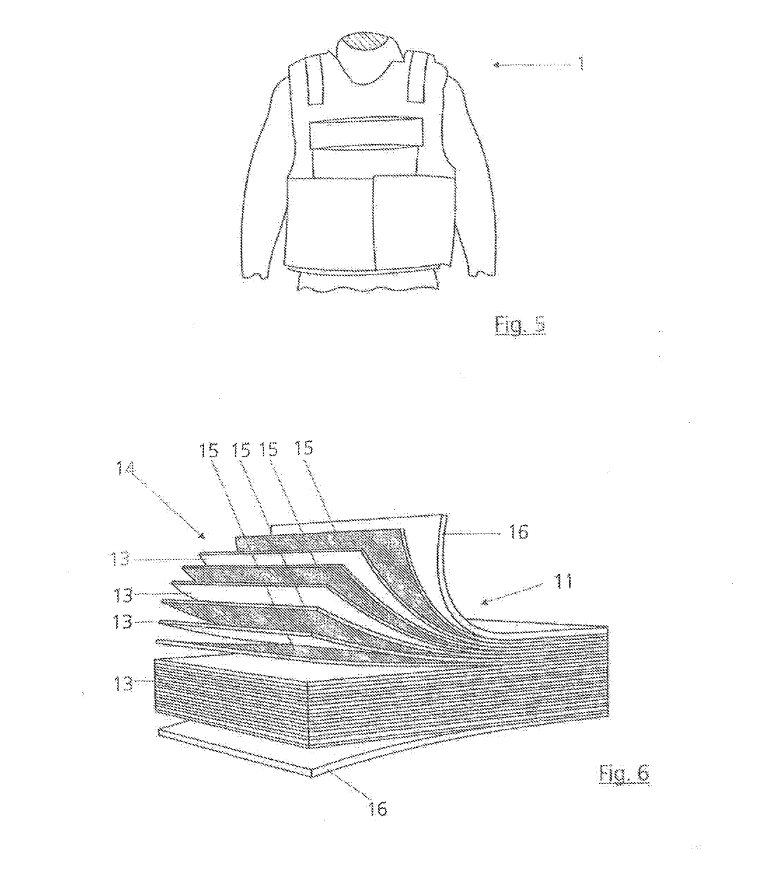

FIG. 6 shows a schematic illustration of a soft-ballistic protective packet employed in the structure of FIG. 5;

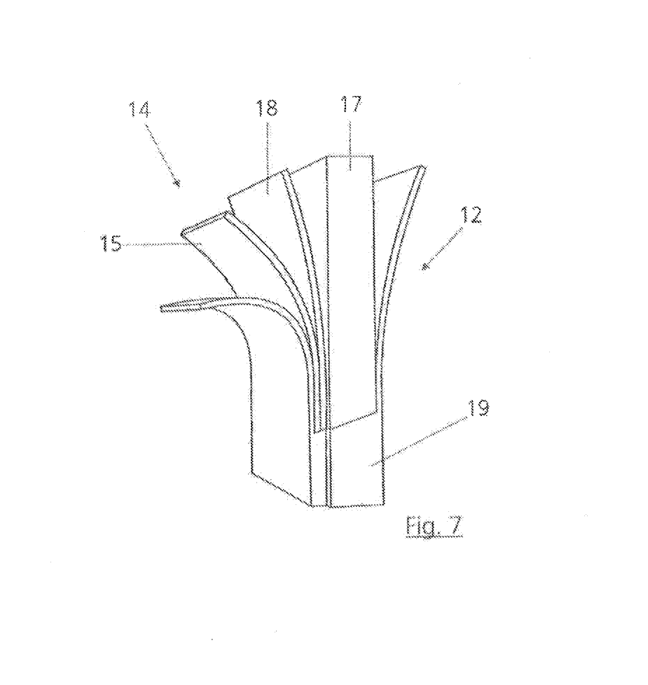

FIG. 7 shows a schematic illustration of a hard-ballistic protective packet employed in the structure of FIG. 5.

The piece of clothing according to the present invention is shown as it would be used with a shirt in FIGS. 1 to 4. The fundamental construction of such pieces of clothing is well known from the general prior art.

It should be understood that in the exemplary embodiment the shirt as provided is windproof, breathable, water-vapor permeable, and watertight.

The piece of clothing 1 is implemented as a protective vest as best seen and understood in FIG. 5.

A protective packet is shown in an embodiment of the invention as a soft-ballistic protective packet (FIG. 6); and in another embodiment as a hard-ballistic protective packet (FIG. 7). Such protective packets are fundamentally known from the general prior art, because of which only the specific embodiment of the protective packet incorporating features of the invention will be described in greater detail, below.

The features which are described, hereinafter, with respect to FIGS. 1 to 4 and 5 to 7 are fundamentally exchangeable, i.e., the features as described, below, are usable in both embodiments (shirt and protective vest).

As should be obvious from a study of FIG. 1, the piece of clothing 1 for a human body, in the present case for a human upper body or torso, has sensors 2, which detect external effects, and relay a signal to an analysis unit 3, and which is schematically illustrated in FIG. 2.

FIG. 1 only shows the sensors 2 in the region of the sleeves of the piece of clothing 1 for reasons of clarity. However, it is provided that all zones 4 (4b, 4c, 4d, 4e, 4f) as explained in more detail hereinafter are provided with the sensors. The zones 4 were not, additionally, illustrated with sensors 2 solely to show the course or region of the different zones.

The sensors 2, which are shown in FIGS. 1 to 4, can be implemented in a first, exemplary embodiment, as electrical circuits. Fundamentally, the sensors 2 can also be implemented as an arbitrary electrical network; as pressure-sensitive sensors; or as piezo elements.

As shown in FIG. 1 and FIG. 3, the electrical circuits 2, or their printed conductors, are implemented in multiple zones (4A-4F respectively), which are assigned to various vital organs. Only four zones 4 are shown for reasons of clarity in FIG. 3. However, the zones 4 which are shown in FIG. 1, are preferably also implemented in the exemplary embodiment according to FIG. 3.

In the exemplary embodiment according to FIG. 1, one zone 4a for the heart, one zone 4b for the two lobes of the lungs, one zone 4c for the stomach, one zone 4d for the liver, two zones 4e for the kidneys, and one large zone 41 for the intestines are provided. The zones 4 can mutually overlap.

FIG. 2 shows an embodiment in which the electrical circuits 2 or their printed conductors cover the entire piece of clothing 1. The implementation of specific zones 4 was omitted.

In FIG. 2, a schematically illustrated power supply is provided in the form of a battery 5. This can be utilized for all embodiments.

As an alternative to the battery 5 as shown, the use of a film battery, is preferably printed on the piece of clothing 1, can also be utilized.

Instead of, or in addition to a battery 5, it can also be provided that a corresponding power supply is generated by the wearer of the piece of clothing 1 himself (as shown).

The electrical circuits 2 communicate with an analysis unit 3, also shown in FIG. 3, and which relays electrical signals using wires or wirelessly to a computer unit 6. The computer unit 6 can preferably relay the information wirelessly to a central monitoring unit, for example, a central action computer 7. A radio unit 8, and/or an interface, in the exemplary embodiment a USB interface 9, can also be provided to relay the signals, as should be obvious from FIG. 3.

The analysis unit 3 can, in one form of the invention, be an integral component of the 35 computer unit 6.

In the exemplary embodiment, the USB interface 9, and the radio unit 8 are integral components of the piece of clothing 1. It can be provided that the radio unit 8 relays the signals directly to an analysis unit which is farther away or communicates with an analysis unit 3, or a computer unit 6, which is fastened to the body of the person who wears the piece of clothing 1, for example, in the form of a backpack.

In the exemplary embodiment, it is provided that the electrical circuits 2 continuously transmit information about the status of the wearer to an analysis unit 3, and optionally to the central action computer 7.

As shown in FIG. 4, in addition to the electrical circuits 2, a vital organ sensor 10, which monitors the heartbeat, for example, is provided in the exemplary embodiment. The vital organ sensor 10 is integrated into the piece of clothing 1 in the region of the heart of a user (not shown).

In a way not shown in greater detail, it can also be provided that the electrical circuits 2 are arranged between two layers, or the electrical circuits 2 are sheathed so that unintentional damage of the electrical network which the printed conductors of the various electrical circuits 2 span is prevented. It can be provided, for example, that the sheath is part of a coating which simultaneously produces the water tightness of the piece of clothing 1.

In a way not shown in greater detail, it can also be provided that the electrical circuits 2 are used in combination with piezo elements or piezoelectric elements. The piezoelectric elements 2 and the electrical circuits 2 can preferably be used in two layers, preferably one on top of another, in the piece 35 of clothing 1, so that both pressure loads or pressure waves and also destruction of the sensors 2 by firearms or stabbing weapons are readily recognized.

In a way not shown in greater detail, the sensors 2 can be arranged on a film both in an embodiment as electrical circuits, and also in an embodiment as piezo elements.

This may be implemented particularly simply if the sensors 2 are implemented as electrical circuits. In this case, the electrical circuits can be printed on the film. In an embodiment of the sensors as piezoelectric elements, it can be provided that they are implemented as a piezo film.

It can be particularly advantageous if the sensors 2 are implemented as electrical circuits, and are printed or applied directly on the piece of clothing. It can also be provided that the electrical circuits are located between two textile plies of the piece of clothing 1 or are printed onto the piece of clothing 1, so that unintentional damage of the printed conductors of the electrical circuits is substantially prevented.

The zones 4 which are shown in the drawings are implemented in the exemplary embodiment on both the front side, and also the rear side of the piece of clothing 1.

In a second embodiment, the sensors shown in FIGS. 1 to 4 can also be implemented as elements 2, whose sensor property is based on a piezo effect. In the exemplary embodiment, these elements are implemented as polymers 2, which have piezoelectric properties which are effected through polarization. In the exemplary embodiment, the polymer 2 is a polyvinylidene fluoride (PVDF). The PVDF is preferably applied in a honeycomb structure or in a meandering form to the piece of clothing 1, as shown in FIGS. 1 to 4.

The features, and variants of the present invention, as described, above, with respect to the first embodiment of the sensors as electrical circuits 2 can also be implemented in a similar manner in an embodiment of the sensors which are formed from a polymer, preferably a polyvinylidene fluoride 2.

In a way not shown in greater detail, it is provided that a further sensor in the form of an electrically conducting or conductive polymer is applied to the honeycomb structure formed by the polyvinylidene fluoride 2, the polymer preferably being in the exemplary embodiment a polyethylene dioxythiopene (PEDOT). The polyethylene dioxythiopene has a structure made of printed conductors, or a conductor structure, which can correspond in its structure to the structure of the electrical circuit 2, as was described, above, with respect to the first embodiment.

The piece of clothing 1 preferably has a circuit, by which it can be established when an exterior pressure is applied to the sensors based on the operation of the polyvinylidene fluoride 2. In this case, an electrical pulse is transmitted via the printed conductors made of the polyethylene dioxythiopene. Furthermore, the circuit recognizes whether the printed conductors made of the polyethylene dioxythiopene have been damaged or interrupted, and which may be caused by ballistic impact or a through-and-through gun shot, or a stabbing injury can be concluded. It can be advantageous for this purpose if a voltage is applied to the printed conductors made of the polyethylene dioxythiopene, and upon the change or disappearance of which a corresponding signal is generated or a recognition occurs by the circuit. This can be performed, for example, in a simple manner in that the voltage which is generated by the piezoelectric properties of the polyvinylidene fluoride, and the voltage which is applied to the printed conductors to recognize their destruction have different frequencies.

The circuit can be part of the analysis unit or can represent it.

It is advantageous if the printed conductors have connection points, and/or soldering points to which further sensors, and in particular vital organ sensors, can be attached.

The power supply of the system can preferably be performed by printed film batteries, or in the way described hereinafter with respect to FIGS. 5 to 7.

A polymer having piezoelectric properties, as described above, can also be combined with an electrical circuit, or small electrical networks, as were described, earlier, with respect to the first embodiment.

The implementation of the piezo elements based upon a polyvinylidene fluoride or a polymer, in general, is also suitable for use in a protective vest. A protective vest is shown as an example hereinafter in FIGS. 5 to 7. An implementation employing printed conductors based upon a polyethylene dioxythiopene or an electrically conductive polymer, in general, is also suitable for use in protective vests.

FIG. 5 schematically shows a protective vest 1, whose construction is fundamentally known from the general prior art. The protective vest 1 has a front side, which is armored, or provided with ballistic protective packets 11, 12, and optionally, a similarly implemented rear side. The rear side can additionally be provided with carrier elements to transport baggage, for example, a backpack, which can simultaneously also have a computer unit 6. Furthermore, it can be provided that the backpack contains a battery. However, it is preferably provided that the battery is a component of the protective packet 11, 12 (shown in greater detail in FIGS. 6 and 7).

FIG. 6 shows a soft-ballistic embodiment of the present invention, and which is incorporated into a protective packet 11. Soft-ballistic bullet-resistant composites, or so-called soft-ballistic protective packets are well known from the general prior art, because of which only the features essential for the invention will be discussed in greater detail hereinafter.

The bullet-resistant composite 11 which is shown in FIG. 6 is formed from multiple plies 13 of a fabric, and which are connected to one another, and in the present case, from multiple plies of a textile material. In the exemplary embodiment, 20 to 40, and preferably 28, plies, 13, of the textile material are provided. The textile material can also be an arbitrary textile material which is suitable to achieve the desired bullet-resistant effect in the composite, and simultaneously remains flexible. Aramid, e.g., aromatic polyimide fibers, are particularly suitable for this purpose.

In the embodiment as shown in FIG. 6, it is provided that an energy accumulator 14 is formed from multiple energy accumulator plies 15, and is integrated in the bullet-resistant composite 11. The energy accumulator plies 15 are each arranged between aramid plies 13. It can be provided that at least one aramid ply 13 is arranged between two energy accumulator plies 15. In the exemplary embodiment, it is provided that there is precisely one ply 13 in this case. In the exemplary embodiment, four energy accumulator plies 15 are typically provided, and which are each arranged and separated by aramid plies 13, and are further located on the side facing toward the body in the bullet-resistant composite 11.

In the soft-ballistic embodiment of the bullet-resistant composite 11 the energy accumulator 14 is located behind the soft-ballistic protective packet, i.e., on the side of the bullet-resistant composite 11 facing away from the outer side. The sensors 2 according to the present invention are also arranged on this side. In the exemplary embodiment, it can be provided that the sensors 2 are integrated in a sheath-shaped film 16, which is preferably watertight, or are arranged thereon. The arrangement is preferably performed on the side of the sheath 16 which faces toward the user's body. Alternatively, a film or a ply which contains the sensors 2 can also be integrated directly into the bullet-resistant composite 11. Preferably this integration can take place in a region adjoining the body of the wearer, for example, between the last energy accumulator ply 15 and the outer sheath 16.

In a way not shown in greater detail, it can be provided that the bullet-resistant composite 11 as shown in FIG. 6 is provided with an additional puncture safeguard, and/or splinter safeguard.

The energy accumulator plies 15 can also be implemented or include in the exemplary embodiment very thin film batteries.

The sensors 2 can be implemented as pressure-sensitive sensors, as piezoelectric elements, as electrical circuits, or as small electrical networks. A combination of the electrical circuits with the piezoelectric elements, or the pressure-sensitive sensors is also possible. It is advantageous if the sensors 2, and in particular in an embodiment which employs electrical conductors, are applied to the UV protective film of the soft-ballistic protective packet 11 or integrated therein. This is possible in a simple manner in particular in that the electrical circuits are implemented as printed circuits.

FIG. 7 shows the embodiment of a hard-ballistic protective packet.

The hard-ballistic composite 12 which is shown in FIG. 7 also has an energy accumulator 14. This is also arranged on the side of the bullet-resistant composite 12 facing toward the body. i.e., the energy accumulator 14 is located in the hard ballistic embodiment behind the so-called “strike face”. The bullet-resistant composite 12 has a rigid, bullet-resistant plate 17, which is connected to the energy accumulator 14. The plate 17 can consist of greatly varying ceramics (preferably high-performance ceramics), polymers, polyethylenes, metals, or a combination of the above-mentioned materials. In the exemplary embodiment, the rigid plate 17 is implemented as a ceramic plate (preferably made of boron carbide). The rigid plate 17 is connected to multiple plies of a fabric 18. The fabric is preferably aramid 18, or a similar material. In the exemplary embodiment, the ceramic plate 17 is connected to multiple plies of aramid 18, which is initially provided in a soft or flexible form as a fabric/scrim, and then permanently, non-removably, and rigidly bonded by means of an adhesive film, or adhesives (not shown in greater detail) to the ceramic plate 17 under the action of pressure and temperature. The ceramic plate 17 is fabricated to also withstand so-called long core gunshots.

In the exemplary embodiment, 30 to 40, and preferably 36, aramid plies 18 are typically provided.

It is provided that the energy accumulator 14 has at least one energy accumulator ply 15. Only one energy accumulator ply 15 is shown in the exemplary embodiment.

According to FIG. 7, it is provided that the energy accumulator ply 15 is non-removably, and rigidly glued to the ceramic plate 17.

The bullet-resistant composite 12 is enclosed by an external sheath 19. The external sheath 19 can be a film, which has the property which was already described with respect to the exemplary embodiment according to FIG. 6. The external sheath 19 is preferably formed from a rubberized material, which encloses the composite 12. A so-called “box solution” can also be provided, in which a preferably rigid or solid box is provided, in which the composite 12 is inserted, and which is subsequently closed by a cover. The composite 12 can be fixed arbitrarily in the box, and preferably embedded or glued.

The arrangement of the sensors 2 can be performed in the hard-ballistic protective packet in the way which was already described with respect to the embodiment according to FIG. 6.

In the embodiment according to FIGS. 6 and 7, the arrangement of an energy accumulator 14 or the arrangement of energy accumulator plies 15 can also be omitted.

What I claim is:

1. A piece of clothing for a human being, comprising:
a first sensor made integral with a piece of clothing, and which is worn by a human body, and wherein the piece of clothing has a zone which overlies a vital organ of the human body when the piece of clothing is worn correctly, and wherein the first sensor is made integral with the zone, and wherein the first sensor is formed, at least in part, of a polyvinylidene fluoride (PVDF) structure which has piezoelectric properties, and which further is capable of generating a first electrical signal which corresponds to a pressure which is applied to the first sensor by an exterior force, and wherein the first electrical signal has a given first frequency;
a second sensor made integral with the zone of the clothing, and which further overlies the vital organ of the human being, and wherein the second sensor is formed, at least in part, of an electrically conductive polymer which defines an electrical circuit, and wherein the electrically conductive polymer is applied in a manner where the second sensor is oriented in at least partial, covering relation relative to the first sensor, and wherein the first sensor is electrically coupled to the electrical circuit, and the first electrical signal is transmitted by the electrical circuit;
a source of electrical power coupled to the electrical circuit, and wherein a second electrical signal having a given frequency is generated by the second sensor when the electrical circuit is partially destroyed by a breach which is formed through the piece of clothing by the exterior force, and wherein the frequency of the second electrical signal is different than the frequency of the first electrical signal, and which is generated by the first sensor; and
an analysis unit coupled in signal receiving relation relative to the first and second electrical signals which are transmitted by the electrical circuit, and wherein the analysis unit distinguishes, based upon the frequencies of the first and second electrical signals, the amount of the exterior force applied to the piece of clothing; the type of destruction experienced by the piece of clothing; and the location of the breach which is formed through the zone of the piece of clothing, and which overlies the vital organ of the human being wearing the piece of clothing.

2. A piece of clothing as claimed in claim 1, and wherein the first and second sensors include multiple first and second sensors which are positioned in multiple discrete zones which are individually assigned to specific vital organs of the human body.

3. A piece of clothing as claimed in claim 1, and wherein the electrically conductive polymer is fabricated from polyethylene dioxythiophene (PEDOT).

4. A piece of clothing as claimed in claim 1, and further comprising a radio unit which is coupled in first and second electrical signal receiving relation relative to the piece of clothing.

5. A piece of clothing as claimed in claim 1, and wherein the first sensor having the PVDF structure comprises a piezo film which is made integral with the piece of clothing.

6. A piece of clothing as claimed in claim 1, and wherein the second sensor is printed directly onto the piece of clothing, and is further disposed, at least in part, in overlying relation relative to the first sensor.

7. A piece of clothing as claimed in claim 1, and wherein the source of electrical power comprises a battery which is made integral with the piece of clothing.

8. A piece of clothing as claimed in claim 1, and wherein the electrical circuit has at least one electrical connection point for electrically coupling yet another sensor for monitoring a biological function of the human body.

9. A piece of clothing as claimed in claim 8, and wherein the biologic function of the human body is selected from the group comprising a heartbeat; a pulse; a respiratory frequency; an oxygen saturation in the blood of the human body; a body temperature; and or a blood pressure.

10. A piece of clothing as claimed in claim 1, and wherein the piece of clothing is a soft-ballistic or hard-ballistic protective vest.

11. A piece of clothing as claimed in claim 10, and wherein the soft-ballistic or hard-ballistic protective vest is formed of multiple soft or hard ballistic protective packets, and wherein the first and second sensors are made integral with an external sheath which encloses the soft or hard-ballistic protective packets.
